(19) United States
(12) Patent Application Publication
(10) Pub. No.: US 2020/0227173 A1
(43) Pub. Date: Jul. 16, 2020

(54) Methods and Systems for Monitoring and Understanding Health Events

(71) Applicant: de Cola Industries LLC, Gainesville, FL (US)

(72) Inventor: Briana de Cola, Gainesville, FL (US)

(21) Appl. No.: 16/742,782

(22) Filed: Jan. 14, 2020

Related U.S. Application Data
(60) Provisional application No. 62/792,326, filed on Jan. 14, 2019.

(51) Int. Cl. G16H 50/30; G16H 40/67

(52) U.S. Cl. CPC G16H 50/30; G16H 40/67

(57) ABSTRACT

The present disclosure relates to a method and system for monitoring and understanding health events in real-time combined with ecological validity. Ecological validity comprises the extent to which the findings of a research study are able to be generalized to real-life settings. More importantly, a method is needed that utilizes objective measures and combines them with real-time user input be able to consistently provide reliable insights for both user and medical professionals. A healthcare provider may then use the descriptions and health data to more deeply understand the causes, effects, and possible mitigating factors for a health event and a user.

### CROSS-REFERENCE TO RELATED APPLICATION

This application claims priority to and the full benefit of U.S. Provisional Patent Application Ser. No. 62/792,326, filed Jan. 14, 2019, and titled “METHODS AND SYSTEMS FOR MONITORING AND UNDERSTANDING HEALTH EVENTS”, the entire contents of which are incorporated in this application by reference.

### BACKGROUND OF THE DISCLOSURE

Mental illness is common throughout the United States, affecting tens of millions of people every year, or approximately 1 in every 5 adults. According to the National Institute of Mental Health, there were an estimated 44.7 million adults with some form of mental illness as of 2016. Globally, 275 million people were diagnosed with anxiety disorders in 2016, while approximately 40 million adults in the United States experienced an anxiety disorder. Only 36.9% of those suffering receive treatment, an overwhelming number when compared to approximately 95,000 actively practicing psychologists.

Anxiety disorders are the most prevalent mental health condition, present in up to 18% of adults in the United States, which account for approximately 42 million adults between the ages of 18 and 54. Anxiety disorders cost the United States more than $42 billion a year, almost one third of the $148 billion total mental health bill for the United States. Despite this prevalence and impact, these disorders do not receive the same recognition as other syndromes such as mood and psychotic disorders. Ten years ago, a primary care physician was usually the primary assessor and treatment provider for anxiety disorders. The diagnosis methodology for anxiety disorders are continuously revised. Today, people with an anxiety disorder are 3 to 5 times as likely to go to the doctor and 6 times more likely to be hospitalized for psychiatric disorders than non-sufferers.

Despite recent technological advancements, the only form of clinical assessment still relies on pen-and-paper evolutions, clinician interviews, and clinical history to treat individuals. Often, a medical professional may request that a patient keep track of their symptoms, moods, sleep patterns, and experiences with medications. There are various methodologies for doing this, such as keeping a mood or anxiety chart, though most fall on the patient to keep track of their symptoms. There are other variables a patient may need to document, such as their triggers, coping techniques, or anything else that might relate to their condition. Though virtual reality (“VR”) has become popular as a therapy tool, this is still assessing triggers of individuals in a clinical setting with little ecological validity. Further, with VR, the clinician generates manufactured triggering environments, which typically source from a limited pool of variables.

As a result, this means that most of the information a medical professional relies on is self-identified and self-reported. A patient coping or addressing any type of mental health condition may not have the wherewithal, the discipline, or the training to track the potential variables associated with their condition and symptoms. Moreover, they may not be able to discern on their own what might be worth reporting. Even the most diligent patient may have lapses in documentation that could be critical to a medical professional.

While there have been strides integrating technology into assessing and diagnosing mental health conditions, there is still a need to increase the reliability of the information shared between a patient and a mental health professional. This would enhance and expedite the creation of an effective treatment for a patient. For anxiety disorders, for example, this would mean developing more effective cognitive behavioral therapy for a patient or determining when selective serotonin reuptake inhibitors may be appropriate.

### SUMMARY OF THE DISCLOSURE

What is needed is a method and system for monitoring and understanding health events in real-time combined with ecological validity. Ecological validity comprises the extent to which the findings of a research study are able to be generalized to real-life settings. More importantly, a method is needed that utilizes objective measures and combines them with real-time user input to be able to consistently provide reliable insights for both user and medical professionals.

In some embodiments, a system may determine what could be a health event, noting any circumstances surrounding a health event, such as what happened leading up to the health event and what happened after. In some implementations, a user may give information that the system stores and weighs based on the user's history with certain health events.

In some aspects, the system may ask questions in real-time that assess what a user is experiencing during a health event. Over time, the system may convey this information to the user or a trained medical professional to distill days, weeks, months, or years of health events into digestible information. This combination of real-time user input with health monitoring may address the self-reporting issues that arise during treatment.

In some embodiments, the system may couple with a health event monitor to maximize its ability to constantly incorporate feedback on a user's state. In some implementations, a user may identify or categorize certain health events for the system. In some aspects, the system may begin to pre-select and sort health events based on a user's history. In some embodiments, the system may make recommendations or remind users about their treatment when the system identifies recurring health events.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates an exemplary health event tracking system, according to some embodiments of the present disclosure.

FIG. 2 illustrates an exemplary communication and data flow diagram, according to some embodiments of the present disclosure.

FIG. 3 illustrates an exemplary communication flow diagram for a health event tracking system, according to some embodiments of the present disclosure.

FIG. 4A illustrates an exemplary health monitor vest, according to some embodiments of the present disclosure.

FIG. 4B illustrates exemplary health monitors, according to some embodiments of the present disclosure.

FIG. 4C illustrates exemplary accessory health monitors, according to some embodiments of the present disclosure.

FIG. 5 illustrates an exemplary graphical user interface (GUI) for a health event tracking system, according to some embodiments of the present disclosure.

FIG. 6 illustrates exemplary method steps for monitoring and identifying trigger events, according to some embodiments of the present disclosure.

FIG. 7 illustrates exemplary method steps for monitoring and identifying trigger events, according to some embodiments of the present disclosure.

FIG. 8 illustrates exemplary method steps for monitoring and identifying trigger event, according to some embodiments of the present disclosure.

FIG. 9 illustrates an exemplary block diagram of an exemplary embodiment of a portable device, according to some embodiments of the present disclosure.

FIG. 10 illustrates an exemplary processing and interface system, according to some embodiments of the present disclosure.

### DETAILED DESCRIPTION

The present disclosure provides generally for health event tracking and monitoring. According to the present disclosure, a health event tracking system may provide real-time information about a health event for a user. In some aspects, the health event tracking system may prompt a user to provide a description of her current activity during or even before a health event. A healthcare provider may then use the descriptions and health data to more deeply understand the causes, effects, and possible mitigating factors for a health event and a user.

In the following sections, detailed descriptions of examples and methods of the disclosure will be given. The description of both preferred and alternative examples though thorough are exemplary only, and it is understood that to those skilled in the art variations, modifications, and alterations may be apparent. It is therefore to be understood that the examples do not limit the broadness of the aspects of the underlying disclosure as defined by the claims.

### Glossary

Health Event: as used herein refers to a predefined health need or crisis that may need to be addressed. In some embodiments, a health event may comprise a symptom of a health condition or disease, such as anxiety, anorexia, or post-traumatic stress disorder, as non-limiting examples. In some aspects, the health event may comprise a symptom of a basic health need, such as hunger, thirst, or exhaustion, as non-limiting examples.
Pre-event: as used herein refers to a period of time before the event where a user may exhibit conditions that are known or likely to precede the occurrence of a health event.
Non-event: as used herein refers to conditions that fall outside of the health event or pre-event. A non-event may be specific or random. In some aspects, a specific non-event may pertain to health but not be directly related to the health event. In some embodiments, a random non-event may not pertain to a defined set of health parameters but may simply fall outside the parameters of a health event or pre-event.
Trigger Event: as used herein refers to any event that may trigger a prompt to a user to input event data. In some aspects, a trigger event may comprise one or more health event, pre-event, and non-event. In some embodiments, the trigger events may be set by one or both the user and the healthcare provider. For example, the healthcare provider may identify the health events and set between five and ten non-events, and the user may be able to select a non-event type.
Health Event Monitor: as used herein refers to a biodevice that receives health data from a user. In some aspects, a health event monitor may comprise a consumer device, such as a smart watch, or a medical-grade device, such as a glucose monitor.
Health data: as used herein refers to data collected by a health event monitor and transmitted to the health event tracking system.
Health parameters: as used herein refer to types and ranges of trackable variables related to a user's health. In some aspects, the health parameters may be based on the trigger event and further refined by one or more healthcare providers, users, or the health event tracking system.

In some embodiments, a health event tracking system may provide real-time information about a health event for a user. Retrospective recall about health events often overlook or incorrectly remember the details surrounding the event. Other issues with pure self-reporting measures may include response bias, misunderstanding of questions, exaggeration, variance in interpretation of rating scales, social desirability bias, lack of ecological validity, no free response questions, post hoc appraisal of behavior, and construct-driven rather than function-led, as non-limiting examples.

Prompting a user to provide a description of her current activity during or even before a health event will allow for more accurate depictions. A healthcare provider may then use the descriptions and health data to more deeply understand the causes, effects, and possible mitigating factors for a health event of a user. In some aspects, prompting may occur in real time, during a trigger event. In some implementations, the prompt may include trigger event inquiries, such as a description of activity, description of how the user feels, or other questions. For example, a trigger event inquiry may comprise a completely unrelated question that may be intended to reduce the risk of the pre-event evolving into a health event.

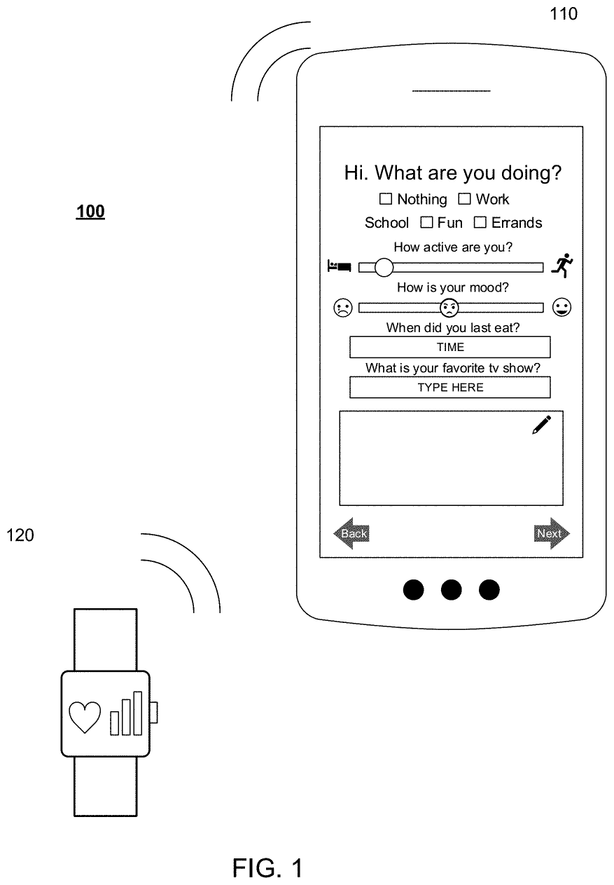

Referring now to FIG. 1, an exemplary health event tracking system 100 is illustrated. In some aspects, a health event monitor 120 may pair with a portable device 110, wherein the portable device 110 may receive health data from the health event monitor 120. In some embodiments, the health event tracking system 100 may monitor for predefined trigger events, each comprising health parameters that may indicate that a user is experiencing a trigger event.

As an illustrative example, a user may be susceptible to anxiety attacks, and the healthcare provider may want to understand the circumstances surrounding a pre-event that may precede an occurrence of the health event of the anxiety attack. Accordingly, the healthcare provider may set the pre-event as a trigger event and may set the health parameters that indicate the pre-event. In some implementations, health parameters may comprise a combination of health parameter types, such as heartrate, temperature, or eye movements, and their respective ranges. In some aspects, the health parameter types may be standard for a trigger event or may be customized based on the user. In some embodiments, the ranges may originally be set based on averages and may evolve based on collected health data.

In some implementations, a health event monitor 110 may comprise a wearable accessory, such as glasses, a watch, or a necklace. In some embodiments, a health event monitor 110 may comprise a medical device, such as a glucose monitor. In some implementations, a health event monitor 110 may be selected based on the monitored health parameters.

In some aspects, the health event tracking system may be able to associate external data with the health data. In some embodiments, the portable device 120 or other devices may provide supplemental data, such as GPS location, date, weather conditions, and app data, as non-limiting examples. When a trigger event is detected, the health event tracking system may be able to store predefined types of external data with the description from the user.

For example, the health event tracking system may be able to note what application the user was engaged with on the portable device. There may be a pattern where heartrate increases when looking at the status of a bank account, anger levels increase when on social media, or paranoia increases when on news outlets. As another example, the health event tracking system may be able to associate the weather conditions with trigger events, such as to determine whether a user may present symptoms of seasonal affective disorder. Where a pre-event may be excessive spending, such as may be associated with hoarding or bipolar, in-app purchases may be tracked and considered with the health data. Exceeding a certain threshold of purchases within a predefined amount of time may trigger a prompt for description.

As another example, GPS may indicate that a user has not left the confines of her house for a week, which may be pertinent information for a healthcare provider to understand her condition. As another example, GPS may indicate that the user is at a huge festival at the occurrence of a health event or pre-event, which may provide objective context to support the description from the user.

In some embodiments, baseline parameters may be used to define the ranges for trigger events. In some aspects, baseline parameters may be directly input or downloaded. In some implementations, the health event tracking system 100 may guide the user through a calibration exercise, which may establish baseline data for one or more health parameters. For example, a user may be prompted to sit, lie down, stand, then walk. In some embodiments, the baseline parameters may be periodically updated or recalibrated.

In some embodiments, as the health event tracking system and healthcare provider receive more health data and descriptions, the health event tracking system may improve. In some aspects, the health event tracking system may accumulate large amounts of health data and descriptions that may be processed into retrain data to continually or periodically update the baseline parameters.

In some embodiments, the portable device 120 may prompt input of a range of inquiries, such as free form text, audio input, visual input, multiple choice, scaled responses, or others, as non-limiting examples. In some aspects, a neutral question, such as “what is your favorite color” or “what is your favorite tv show” may be included as a constant to ensure the user is completing the prompts and the user is in a lucid state. In some implementations, the types of inquiries may be customized to the user. In some aspects, a user may be able to dictate the description. In some embodiments, a user may be able to include photographs, drawings, or video as part of the description.

As an illustrative example, the user may have a learning disability that limits her ability to read, particularly under stress, so her inquiries may not include text instructions. Instead, her prompts may include pictorial representations. As another illustrative example, the health event may relate to controlling her tempter, so her “mood” options may include a scale from calm to furious. Where a user may be dealing with depression with no anger issues, the “mood” options may include a scale from tears to a smile.

As an illustrative example, a young child may be presenting symptoms of autism, and the parents and healthcare providers may want a deeper understanding of the severity and symptoms. The health event monitors may comprise a speaker, an accelerometer, eye tracking device (such as glasses or contact lenses), electrodermal device, tactile device, and sleep monitors. The health parameters may comprise speech and acoustic differences, stereotypical behaviors, gaze patterns, moisture levels, and neurophysiological and cardiorespiratory data. A portable device for the young child may be special for a person with autism, a child, or both. The description prompts may also be specifically tailored to the abilities of a child at that age or to a person with a level of autism. In some embodiments, the prompts may evolve based on health data and descriptions.

In some aspects, a health event may be exacerbated by trigger words or images, and the prompts may be customized to avoid those triggers. As an illustrative example, the health event may be starvation due to an eating disorder, and the pre-events may comprise hunger, thirst, and anxiety, wherein the health parameters may comprise blood sugar levels, heartrate, and others, such as may monitored through ECG, stomach gastric device, mood monitor, and glucose monitor. The prompts for the user may avoid any terms related to eating, weight, or food, which may make the user more acutely aware of their disorder, increasing her anxiety. As another illustrative example, the health events may be symptoms of PTSD caused by domestic abuse. The prompts may exclude any words and images that may remind the user of the abuse.

In some aspects, the prompting may be able to diffuse the progression of a pre-event into a health event, such as by distracting the user or by directed inputs. For example, if the pre-event is becoming overwhelmed, the portable device 120 may be programmed to play the user's favorite song as a notification, which may draw the user's attention away from whatever is overwhelming her. As another example, where the pre-event is stuttering, the portable device may prompt the user to recite a calming sentence.

In some implementations, the health event monitor 110 may use saved or stored images that the user programs into the system. In some embodiments, the user may store specific image, such as images that invoke happy thoughts, or images that may be used to invoke different moods by the user. In some aspects, the user may manually request the images to show, or the portable device 120 may activate a specific image based on a user's response during pre-events, or heart rate, as non-limiting examples.

For example, the user may be in a negative mood which may be a factor in a pre-event. The user may then activate a positive image to be displayed on the screen of the health event monitor. This may then elevate the mood of the user, giving them a more positive behavior or relaxing them from a stressful event. In another example, the portable device 120 may sense a rise in heart rate or pulse and display a saved image that may calm the user down.

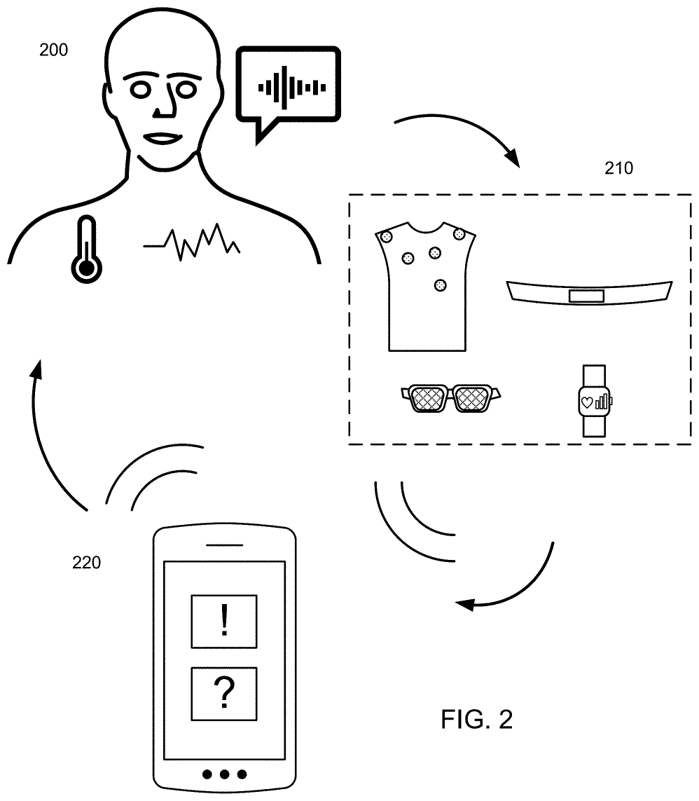

Referring now to FIG. 2, an exemplary communication and data flow diagram is illustrated. In some aspects, health event monitors 210 may track and monitor defined health parameters of a user 200. The monitored health parameters may depend on the health event and may include, loudness, heart rate, or temperature, as non-limiting examples. In some embodiments, the health event monitors 210 may communicate wirelessly with a portable computing device 220, such as a smartphone, tablet, or laptop. In some aspects, the computing device 220 may communicate with the user 200 to prompt a health description. In some implementations, the prompting may occur during a pre-event, a health event, or a non-event, depending on the settings and preferences associated with the user and health event.

In some embodiments, a user 200, her healthcare provider, or both may be able to set the notification parameters. For example, where the health event is exhaustion or hunger, prompting health descriptions during a pre-event may be helpful and may reduce the chance of the user 200 transitioning to the health event. Awareness of the pre-event may allow the user 200 to prevent the health event.

As another example, the health event may be an anxiety attack, and each prompting may increase the risk of escalating the user from a pre-event to the health event. There, promptings during non-events may reduce the anxiety associated with the prompting. The healthcare provider may inform the user 200 that the health event tracking system may prompt random descriptions throughout the day, without specifically stating the conditions that may trigger the prompt. Where the prompts only occur during pre-events, the user 200 may deduce what triggers the prompt, which may increase the anxiety associated with the prompting. Where the prompts also occur during non-events, the user 200 may not anticipate an anxiety attack.

As another example, a user may present with obsessive compulsive disorder, and the health event may be obsessive motions that may be excessively repetitive and accompanied by increased heartrate. An accelerometer may be useful to track the motions and a heartrate monitor may track the heartrate. For example, some smart watches comprise both a heartrate monitor and an accelerometer.

As another example, the user 200 with schizophrenia may experiences hallucinations or delusions. Prompts may ask for descriptions of what the user is seeing, hearing, smelling, tasting, and even touching in real-time. An accelerometer may also track when the user is in a catatonic state, where the user may not move or be responsive to prompts for extended periods of time.

In some aspects, the health event monitors may comprise one or more sensors that may track one or more health parameters. As non-limiting examples, the sensors may comprise temperature sensors, proximity sensors, accelerometers, IR sensors, pressure sensors, light sensors, ultrasonic sensors, chemical sensors (such as for smoke, gas, or alcohol).

In some implementations, the health event monitor 210 may have the ability to track muscle growth and certain tendon activity. In some aspects, the health event monitor 210 may use patterns in muscle activity to alert the user of a possible growth issue. In some embodiments, there may exist a system that may monitor the muscle growth activated by certain activities the user is doing.

For example, the health event monitor 210 may track the user's muscle movement and overall usage using a sensor. In some embodiments the health event monitors 210 may recognize a pattern in overcompensation of a muscle group. The monitor may then alert the user of a possible tendon tear or muscle tear based on automated patterns installed by physicians.

In some embodiments, the health parameters may include a range of types. As non-limiting examples, the types of health parameters may include heartrate, blood sugar, electrodermal activity, cortisol level, ECG, eye movements, vocal loudness, vocal stability, temperature, moisture levels, chemical levels, body motion, mouth dryness, or jaw tension. In some aspects, the health parameters for a health event may comprise a combination of types. For example, the health parameters for a health event related to an anxiety attack may include heartrate ranges, breathing patterns, and skin moisture. In some implementations, the health parameters may be monitored by one or more health event monitors.

As an illustrative example, a user may be dealing with alcoholism. The health event may be consuming alcohol, and pre-events may comprise dry mouth, stress, anxiety, and the shakes. Accordingly, the health event monitors may sense moisture levels in the mouth, jaw tension, heartrate, and body movements. The body movements may be monitored through accelerometers that may be able to determine the fluidity and motion of the movements, wherein quick, repetitive motions over a minute may fall within the ranges for a pre-event.

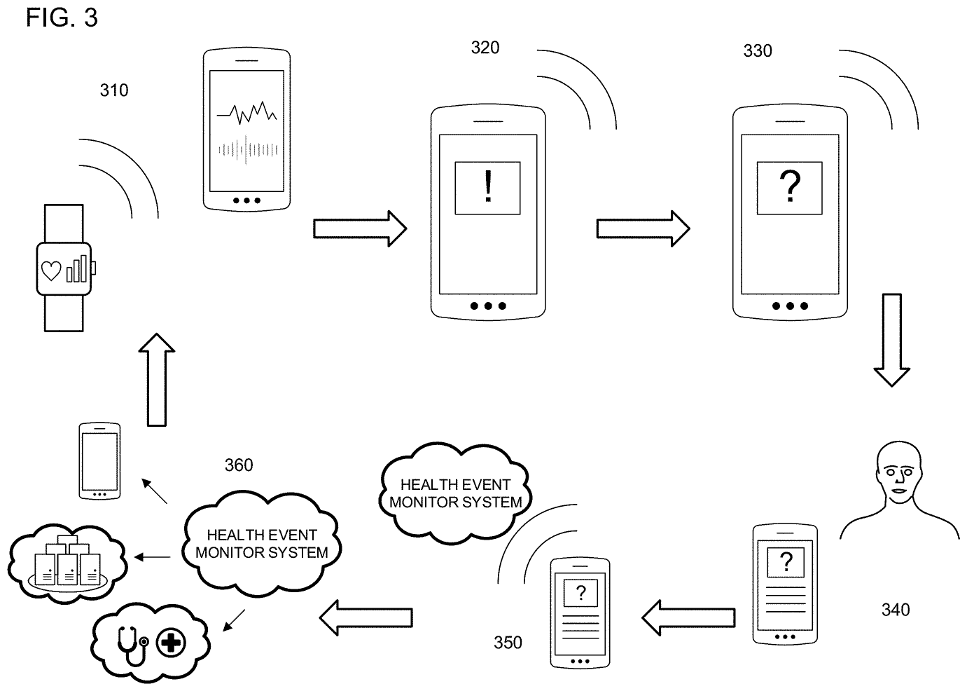

Referring now to FIG. 3, an exemplary communication flow diagram for a health event tracking system is illustrated. At 310, a health monitor may communicate with a portable device, wherein the health monitor may transmit health data of a user. In some aspects, at 320, a portable device may receive health data that indicates a trigger event, such as a pre-event, health event, or non-event conditions, wherein the portable device may transmit a notification, such as to a user. In some implementations, the notification may comprise an alert, such as a visual, audio, or haptic feedback. In some embodiments, the notification may further comprise alert details, such as affirmations, trigger event types, or trigger event conditions.

For example, a user may set a daily calorie burned amount, the notification may include a statement such as “Great job! You met your daily calorie burning goal!” As another example, the alert may state, “You are getting hungry, don't forget to eat.” In some embodiments, a notification about the type of trigger event may exacerbate the conditions, and the settings may allow for removal of that portion of the notification.

At 330, the portable device may prompt a user to input a health event description. At 340, a user may input a health event description. At 350, the portable device may transmit the health event description, the health data, or the triggering health event, pre-event, or non-event. At 360, the health event monitoring system may transmit the user data to one or more locations, such as to a memory source, a healthcare provider, or to a portable device.

In some aspects, the user data may comprise health data and descriptions, wherein the health data may be stored with a user profile in the memory source, such as a cloud memory.

In some embodiments, at least a portion of the user data may be stored locally. In some implementations, the user data may be transmitted to a healthcare provider system, which may allow a healthcare provider to access the user data. In some embodiments, the healthcare provider may receive notifications or alerts related to the user, such as when new data is available or when a user is suffering from a severe health event.

In some embodiments, a notification may be activated for a timed event, such as a workout or a marathon. For example, the event tracking system may allow for the user to have a preset timed workout plan. If the user forgets the plan all together or misses a step, the system may send the user an automated reminder or message to follow the preset steps of their plan.

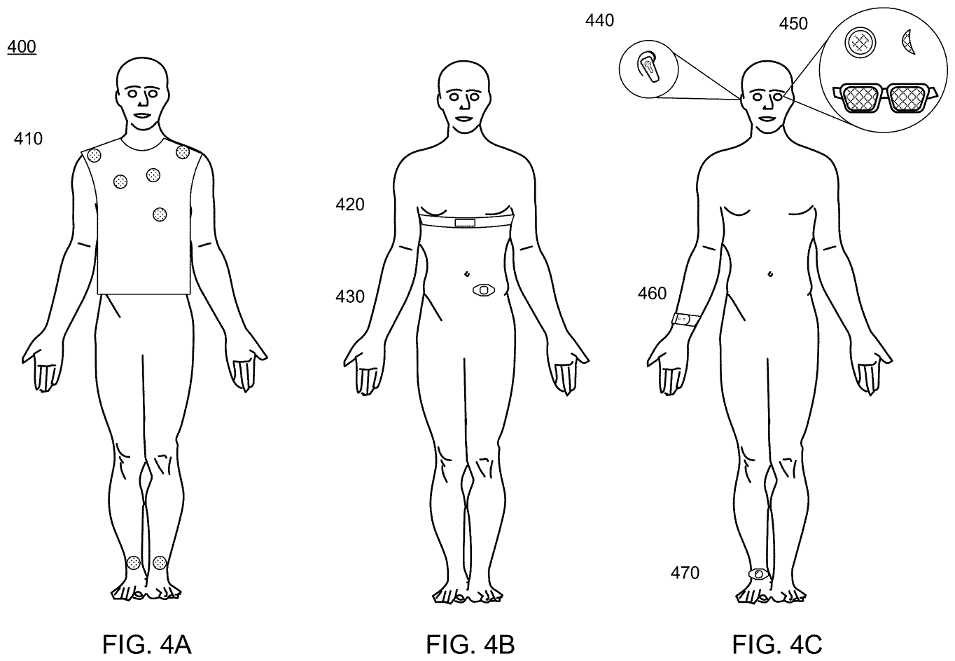

Referring now to FIGS. 4A-4C, exemplary health monitors are illustrated. In some embodiments, a user may wear a health monitor vest 410. In some implementations, the health monitor vest 410 may comprise comfort apparel, such as for fitness. In some aspects, the health monitor vest 410 may be a medical grade product that adapts hospital or healthcare devices into a piece of apparel. For example, the health monitor vest 410 may comprise multiple leads for an ECG. In some embodiments, a user may wear a chest health monitor 420. In some implementations, a user may wear a bio-patch 430 or medical monitoring device, such as may be integrated with an insulin pump.

In some embodiments, a user may wear an ear health monitor 440 that may track auditory and vocal health parameters. In some aspects, a user may wear an ophthalmic device 450, such as glasses or contacts. The ophthalmic device 450 may track voluntary and involuntary eye movements, which may be useful for health events related to users on the autism spectrum, with learning disabilities, and with attention deficit/hyperactivity disorder, as non-limiting examples. In some aspects, the ophthalmic device 450 may monitor biomarkers in the eye. In some implementations, a user may wear an accessory health monitor 460, 470, such as a watch, a necklace, or a ring, as non-limiting examples. In some embodiments, an accessory health monitor 460, 470 may be worn on the arms, hands, legs, and feet.

In some implementations, a user may cycle through different health monitors. For example, initially, a user may utilize a health monitor vest 410, which may allow for highly accurate collection of health data. In some aspects, as the severity or frequency of health events decreases, the user may be able to use a chest health monitor 420 and then eventually an accessory health monitor 430.

Each health monitor may be suited for different needs. For example, a health monitor vest 410 may not be appropriate for extended wear beyond a few weeks. As another example, a chest health monitor 420 may only be appropriate where one of the health parameters comprises a heartrate. The health monitor type may also depend on the level of care needed. For example, where the user may suffer mildly from anxiety, a health monitor vest 410 may be excessive. In contrast, where the user has difficulty functioning, a health monitor vest 410 may be able to collect more and better health data, allowing for a more nuanced understanding of the conditions.

In some aspects, there may exist one interface where all health monitors exist in one database. For example, all of the health monitors may gather their data on to one portable device or application. Rather than collecting data from multiple health monitors, an all-in-one interface may be available.

In some implementations, there may exist a collective average from each health monitor collected to give the user the most accurate depiction of their health. In some embodiments, the database may notice patterns, such as inclines, declines, deficiencies, overcompensation or any other unusual health issues, as non-limiting examples.

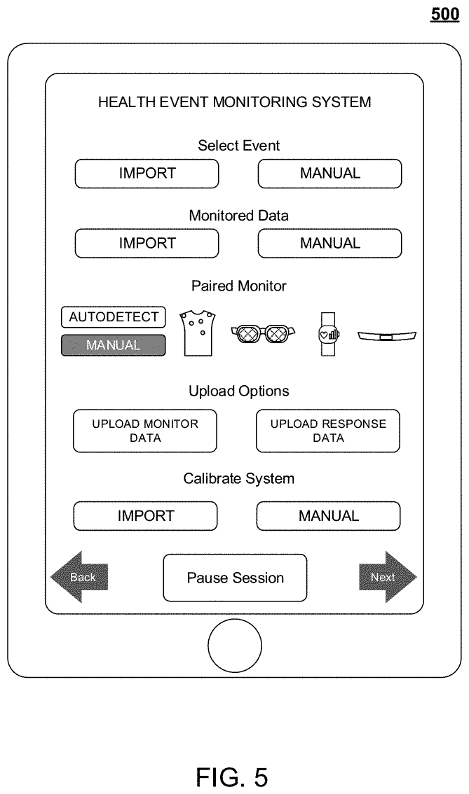

Referring now to FIG. 5, an exemplary graphical user interface (GUI) 500 for a health event tracking system is illustrated. In some aspects, the GUI 500 may allow for the input of health events, such as by manual input or by downloading an external file. In some embodiments, the GUI 500 may allow for the input of monitored data, such as by manual input or by downloading an external file. In some implementations, the external file for the event selection and the monitored data may be the same. In some aspects, the settings may be based broadly on uploaded data and tuned by a manual input. In some implementations, the GUI 500 may allow for the selection of paired monitors. In some aspects, the paired monitors may be autodetected, such as during active pairing. In some implementations, the GUI 500 may allow for upload of one or both monitor data and response data.

In some embodiments, calibration may occur manually, such as directly through the portable device and the monitors. In some aspects, calibration may occur at least in part through downloading baseline health data. In some implementations, the GUI 500 may allow for temporary pause or suspension of a session. A user may want to suspend the tracking where an activity may skew the health data, such as during exercise.

In some aspects, the GUI 500 may be available to one or both the user and the healthcare provider. Where the GUI 500 may be accessible by both, some of the options may not be adjustable for the user. For example, a user may not be able to access or control the calibration or health event portions, but the user may be able to access the suspend session function and selection of non-events, such as number of steps walked per day, calorie intake, or activity levels, as non-limiting examples.

In some embodiments, the healthcare provider may have the ability to set goals for the user or instruct them to follow a certain diet, or exercise plan. In some implementations, the user may provide feedback to the healthcare provider if the workout was too difficult or the diet was not filling, as non-limiting examples. This may provide a better, constant form of communication and feedback between the user and healthcare provider.

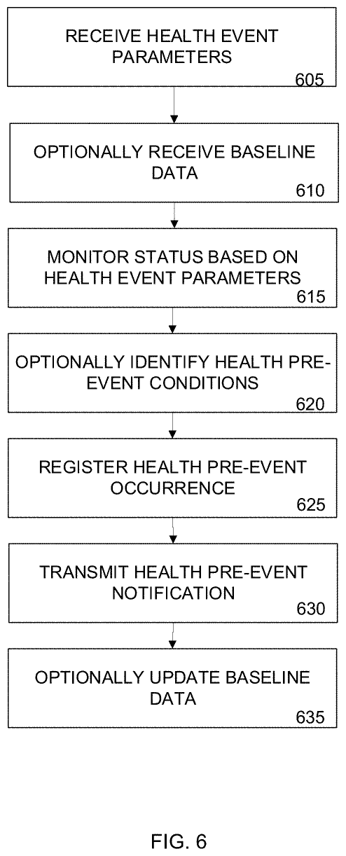

Referring now to FIG. 6, exemplary method steps for monitoring and identifying health events are illustrated. At 605, health event parameters may be received. In some aspects, at 610, baseline data may be received. At 615, based on health event parameters, the status of a user may be monitored. In some implementations, at 620, pre-event conditions may be identified. In some implementations, pre-event conditions may be input directly. At 625, a trigger event occurrence may be registered. At 630, a trigger event notification may be transmitted. In some embodiments, at 635, baseline data may be updated. In some implementations, the baseline data may periodically update as retraining data customized to the actual health data of a particular user.

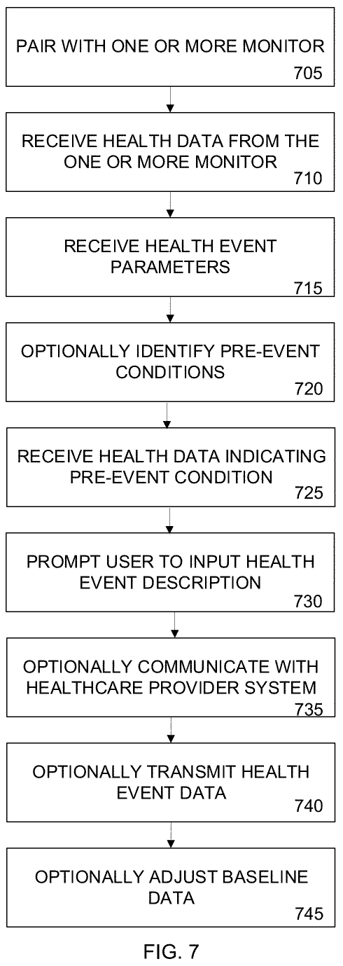

Referring now to FIG. 7, exemplary method steps for monitoring and identifying trigger events are illustrated. At 705, one or more monitors may be paired. At 710, health data may be received from the pair monitors. At 715, health event parameters may be received. In some aspects, at 720, pre-event conditions may be identified. At 725, health data indicating pre-event conditions may be received. At 730, a user may be prompted to input health event descriptions. In some embodiments, at 735, healthcare provider system may receive a communication. In some aspects, at 740, health event data may be transmitted. In some implementations, at 745, baseline data may be adjusted.

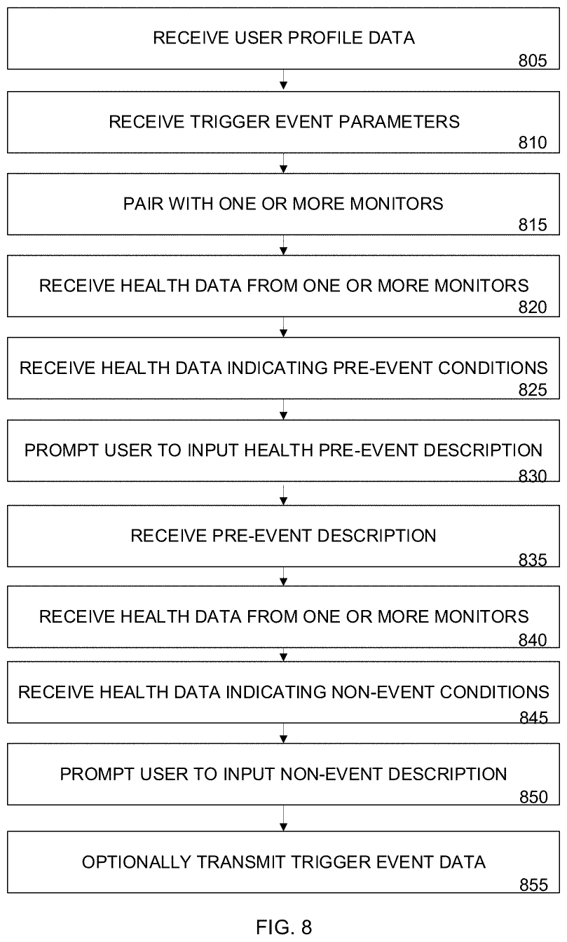

Referring now to FIG. 8, exemplary method steps for monitoring and identifying trigger event are illustrated. At 805, user profile data may be received. In some embodiments, profile data may comprise user preferences, healthcare provider preferences, baseline/calibration data. At 810, trigger event parameters may be received. In some implementations, trigger events may comprise health events, pre-events, and non-events. In some aspects, trigger event parameters may comprise types of health data and ranges of health data, as non-limiting examples. In some embodiments, collected health data and descriptions may be stored with a user profile.

At 815, one or monitors may be paired. At 820, health data may be received from the paired monitors. At 825, health data indicating pre-event conditions may be received. In some aspects, pre-event conditions may be a predefined percentage outside the health event ranges. In some implementations, pre-event conditions may be a separate set of parameters that likely lead to the health event.

At 830, a user may be prompted to input health pre-event descriptions. At 835, pre-event description may be received. In some embodiments, the description prompt may be open, directed, or both. For example, some of the description may be multiple choice, yes or no questions, scaled responses, or free form text. In some aspects, the description prompt may be standard based on the health event, customized by the healthcare provider or user, or combinations. For example, the standard description prompt for any trigger event may allow for freeform text, and the healthcare provider may add more directed prompts based on the needs of the user. In some embodiments, the description prompt settings may provide multiple options and the healthcare provider or user may select from a collection of options.

At 840, health data may be received from the paired monitors. At 845, health data indicating non-event conditions may be received. At 850, a user may be prompted to input non-event description. Prompting descriptions for non-events may reduce the probability of the user developing reactions to the prompts. If every time they receive the prompt, they are about to have an anxiety attack, the prompt may accelerate their anxiety. In some aspects, non-events may be randomized as long as the conditions fall outside the pre-event parameters.

In some implementations, non-events may be specific conditions that may be of interest to the user/healthcare provider. In some embodiments, non-events for one user may be a health event or pre-event for another user. For example, sleepiness may be a non-event for a user with body dysmorphia, a pre-event for a user with bulimia nervosa, and a health event for a user with narcolepsy.

For example, a non-event may be low blood sugar or hunger, and a health event may be increased moisture levels or trembling, which may be symptoms of PTSD. Hunger may not be directly associated with PTSD, but eating patterns may be generally useful to know to ensure the user is properly taking care of herself. In some aspects, the types of non-events may be customizable, each with their own set of parameters and conditions. In some embodiments, the types of non-events may be limited based on the capabilities of the monitors.

In some aspects, at 855, trigger event data may be transmitted, such as to external memory resources or a healthcare provider system, as non-limiting examples. In some aspects, transmitting data related to non-events may be useful to fully understand the health event descriptions. In some embodiments, the number of randomized non-event prompts may be adjustable. For example, the number may be too low if they still cause a spike in anxiety, and the number may be too high if they disrupt the user's day.

In some aspects, health parameters for a trigger event may be received, such as from a user, a healthcare provider, or external system. In some embodiments, health parameters may be identified, such as through analyzing health data associated with a user. In some implementations, the health parameters for trigger events may be based on baseline data. The baseline data may be based on typical health data associated with patients similar to the user, such as a non-smoker, 32 year-old female or a bipolar, 19 year old male. In some aspects, the baseline data may be based on the user. In some embodiments, the baseline data may be adjusted over time based on received health data. Adjusting the baseline data may allow for modification of the health parameters to allow for more accurate monitoring.

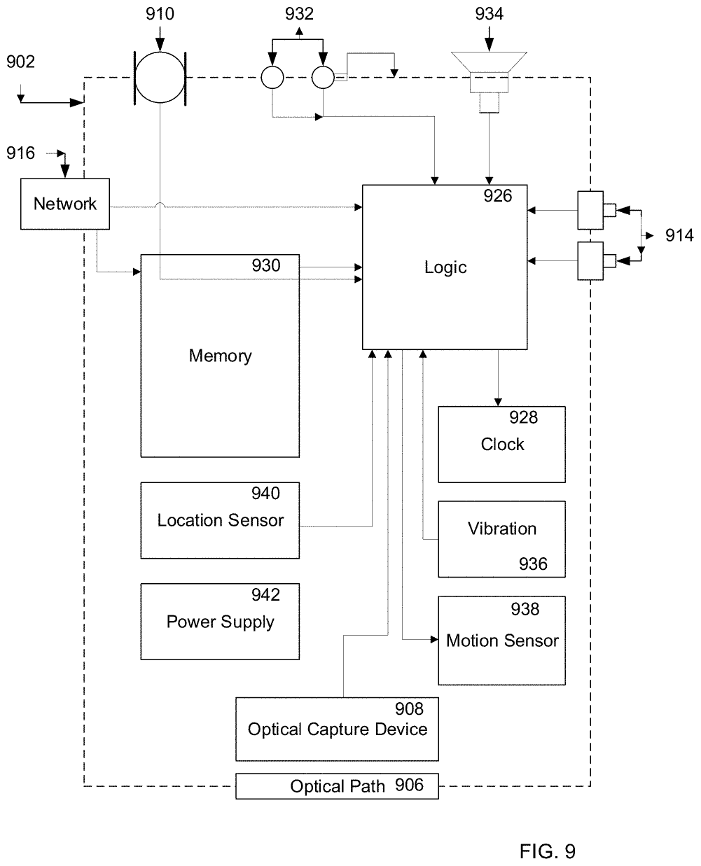

Referring now to FIG. 9, an exemplary block diagram of an exemplary embodiment of a portable device 902 is illustrated. The portable device 902 may comprise an optical capture device 908, which may capture an image and convert it to machine-compatible data, and an optical path 906, typically a lens, an aperture, or an image conduit to convey the image from the rendered document to the optical capture device 908. The optical capture device 908 may incorporate a Charge-Coupled Device (CCD), a Complementary Metal Oxide Semiconductor (CMOS) imaging device, or an optical sensor of another type.

In some embodiments, the portable device 902 may comprise a microphone 910, wherein the microphone 910 and associated circuitry may convert the sound of the environment, including spoken words, into machine-compatible signals. Input facilities 914 may exist in the form of buttons, scroll-wheels, or other tactile sensors such as touchpads. In some embodiments, input facilities 914 may include a touchscreen display. Visual feedback 932 to the user may occur through a visual display, touchscreen display, or indicator lights. Audible feedback 934 may be transmitted through a loudspeaker or other audio transducer. Tactile feedback may be provided through a vibration module 936.

In some aspects, the portable device 902 may comprise a motion sensor 938, wherein the motion sensor 938 and associated circuitry may convert the motion of the portable device 902 into machine-compatible signals. For example, the motion sensor 938 may comprise an accelerometer, which may be used to sense measurable physical acceleration, orientation, vibration, and other movements. In some embodiments, the motion sensor 938 may comprise a gyroscope or other device to sense different motions.

In some implementations, the portable device 902 may comprise a location sensor 940, wherein the location sensor 940 and associated circuitry may be used to determine the location of the device. The location sensor 940 may detect Global Position System (GPS) radio signals from satellites or may also use assisted GPS where the portable device may use a cellular network to decrease the time necessary to determine location. In some embodiments, the location sensor 940 may use radio waves to determine the distance from known radio sources such as cellular towers to determine the location of the portable device 902. In some embodiments these radio signals may be used in addition to and/or in conjunction with GPS.

In some aspects, the portable device 902 may comprise a logic module 926, which may place the components of the portable device 902 into electrical and logical communication. The electrical and logical communication may allow the components to interact. Accordingly, in some embodiments, the received signals from the components may be processed into different formats and/or interpretations to allow for the logical communication. The logic module 926 may be operable to read and write data and program instructions stored in associated storage 930, such as RAM, ROM, flash, or other suitable memory. In some aspects, the logic module 926 may read a time signal from the clock unit 928. In some embodiments, the portable device 902 may comprise an on-board power supply 932. In some embodiments, the portable device 902 may be powered from a tethered connection to another device, such as a Universal Serial Bus (USB) connection.

In some implementations, the portable device 902 may comprise a network interface 916, which may allow the portable device 902 to communicate and/or receive data to a network and/or an associated computing device. The network interface 916 may provide two-way data communication. For example, the network interface 916 may operate according to an internet protocol. As another example, the network interface 916 may comprise a local area network (LAN) card, which may allow a data communication connection to a compatible LAN. As another example, the network interface 916 may comprise a cellular antenna and associated circuitry, which may allow the portable device to communicate over standard wireless data communication networks. In some implementations, the network interface 916 may comprise a Universal Serial Bus (USB) to supply power or transmit data. In some embodiments, other wireless links known to those skilled in the art may also be implemented.

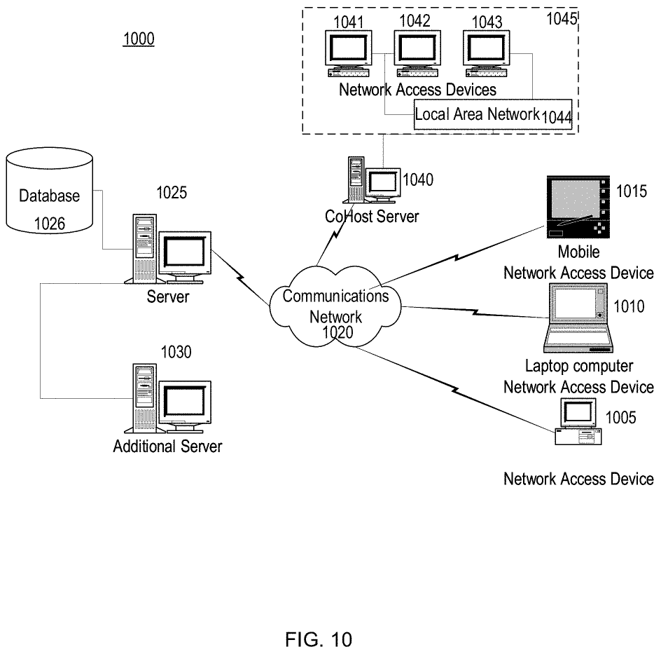

Referring now to FIG. 10, an exemplary processing and interface system 1000 is illustrated. In some aspects, access devices 1015, 1010, 1005, such as a paired portable device 1015 or laptop computer 1010 may be able to communicate with an external server 1025 though a communications network 1020. The external server 1025 may be in logical communication with a database 1026, which may comprise data related to identification information and associated profile information. In some embodiments, the server 1025 may be in logical communication with an additional server 1030, which may comprise supplemental processing capabilities.

In some aspects, the server 1025 and access devices 1005, 1010, 1015 may be able to communicate with a cohost server 1040 through a communications network 1020. The cohost server 1040 may be in logical communication with an internal network 1045 comprising network access devices 1041, 1042, 1043 and a local area network 1044. For example, the cohost server 1040 may comprise a payment service, such as PayPal or a social network, such as Facebook or a health system. In some aspects, the interface system 1000 may comprise one or more processors.

In some embodiments, the database 1026 may comprise one or more memory resources, such as a user profile database or health event database. In some implementations, the health event database may be external and accessible through the interface system 1000. For example, the health event database may be part of a healthcare or hospital system, such as through a permission from a healthcare provider. In some embodiments, the health event database may comprise trigger event data for a plurality of health events, wherein trigger event data may relate to one or more of health parameters, pre-events, health event monitors, and health data.

The trigger event data may provide condition information and identify relevant health data types, wherein monitoring the relevant health data types may allow for identification of a trigger event. A trigger event may comprise a non-event, a pre-event, or a health event. In some aspects, the trigger events may be adjusted in settings, allowing for monitoring for only some trigger events. For example, a psychologist may only want to prompt an event inquiry during a pre-event or health event.

In some aspects, the memory resources may be connectable to devices and systems through a communications network 1020, wherein at least one device comprises a health event monitor and at least one device comprises a display device. In some aspects, a health event monitor may be part of the interface system 1000. In some embodiments, a health event monitor may be an external device.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A system for monitoring health events, wherein the system comprises:
one or more processors;
one or more memory resources comprising:
a user profile database;
wherein the one or more memory resources are connectable to devices and systems through a communications network, wherein at least one device comprises a first health event monitor, at least one device comprises a display device, and at least one system comprises a health event database, wherein the one or more memory resources are executable by the one or more processors to perform the steps of:
receiving health data from at least the first health event monitor;
receiving from the health event database a first set of health event parameters comprising at least one health data type and at least one trigger event, wherein the at least one trigger event comprises an identifiable change in health data;
monitoring health data based on the first set of health event parameters comprising at least one health data type and at least one trigger event, wherein the at least one trigger event comprises an identifiable change in health data;
detecting the at least one trigger event;
prompting the user to input a response to a trigger event inquiry, wherein the prompting and the input occurs through the display device; and
transmitting the response and the health data received during the at least one trigger event to one or both the user profile database and an external system.

2. The system of claim 1, further comprising the first health event monitor.

3. The system of claim 1, further comprising the display device.

4. The system of claim 1, wherein an external system comprises the health event database.

5. The system of claim 1, wherein the health event monitor comprises the display device.

6. The system of claim 1, further comprising the health event database.

7. The system of claim 1, wherein the health event database comprises trigger event data for a plurality of health events, wherein trigger event data relates to one or more of health parameters, pre-events, health event monitors, and health data.

8. The system of claim 1, wherein the prompting occurs in real time during the at least one trigger event.

9. The system of claim 1, further comprising:
identifying the at least one trigger event as a health event, a pre-event, or a non-event based on predefined criteria.

10. A method for monitoring for health events of a user, the method comprising:
receiving health data from one or more health event monitors;
monitoring health data based on a first set of health parameters comprising at least one health data type and at least one trigger event, wherein the at least one trigger event comprises an identifiable change in health data;
detecting the at least one trigger event;
prompting the user to input a response to a trigger event inquiry; and
transmitting the response and the health data received during the at least one trigger event.

11. The method of claim 10, further comprising identifying at least a portion of the first set of health parameters.

12. The method of claim 10, further comprising:
pairing with one or more health event monitors, wherein each of the one or more health event monitors collects health data of a user.

13. The method of claim 10, wherein the prompting occurs in real time during the at least one trigger event.

14. The method of claim 10, further comprising:
identifying the at least one trigger event as a health event, a pre-event, or a non-event based on predefined criteria.

15. The method of claim 10, further comprising:
identifying the first set of health parameters based at least in part on a first health event, wherein the first set of health parameters identify at least one health data type.

16. The method of claim 15, wherein the monitoring further comprises:
extracting relevant health data based on the at least one health data type; and
and analyzing the relevant health data.

17. The method of claim 10, wherein the first set of health parameters are based in part on baseline data.

18. The method of claim 17, wherein the baseline data comprises predefined standard health data.

19. The method of claim 17, wherein the baseline data comprises user-specific health data.

20. The method of claim 19, further comprising:
updating the baseline data according to received health data; and
modifying the first set of health parameters based on the updated baseline data.
